(19) United States
(12) Patent Application Publication
(10) Pub. No.: US 2025/0147015 A1
(43) Pub. Date: May 8, 2025

(54) PROTEIN ANALYSIS PLATFORM AND USE THEREOF

(71) Applicant: National Taiwan University, Taipei (TW)

(72) Inventors: AN-BANG WANG, Taipei (TW); WEI-WEN LIU, Taipei (TW); SI-TSE JIANG, Taipei (TW); CHIA-WEI HSU, Taipei (TW); TING-CHI HUANG, Taipei (TW)

(21) Appl. No.: 18/941,070

(22) Filed: Nov. 8, 2024

Related U.S. Application Data
(60) Provisional application No. 63/597,177, filed on Nov. 8, 2023.

(51) Int. Cl. G01N 33/543; B01L 3/00; G01N 33/68

(52) U.S. Cl. CPC G01N 33/54373; B01L 3/502761; G01N 33/6803; B01L 2200/0652; B01L 2300/069; B01L 2400/0421; G01N 2474/10

(57) ABSTRACT

A protein analysis platform includes a platform body comprising: a gel working unit provided in a top portion of the platform body and comprising a gel accommodation area for accommodating at least one gel; at least one electrophoresis tank provided along a side of the gel accommodation area and provided with at least one electrode; and a blotting layer stack provided in a bottom portion of the gel working unit and comprising an electrode layer; wherein a removable bottom plate is provided between the gel working unit and the blotting layer stack and detachably corresponds to a bottom side of the gel accommodation area. The protein analysis platform is used for western blotting or next-generation western blotting, wherein the protein analysis platform can quickly complete steps such as gel casting, electrophoresis, and blotting in one platform.

### BACKGROUND OF THE INVENTION

### 1. Technical Field

The present invention relates to protein analysis apparatus. More particularly, the invention relates to, but is not limited to, a protein analysis platform that integrates gel casting, electrophoresis, and blotting, and a use of the protein analysis platform.

### 2. Description of Related Art

Recent years have seen a rising demand in the biochemical testing market and an increasing variety of biochemical testing applications, especially those related to medical testing. However, rapid and consistent provision of reliable test results remains a long-term, unsolved challenge. In the post-pandemic era, in particular, screening techniques have become a major target of research in the field of public health worldwide.

The conventional western blot method is rather complicated and generally involves three discontinuous steps performed with three separate apparatus, including making a gel in a gel casting device, taking out the gel after it has taken shape and mounting it in an electrophoresis device, and then transferring the gel to a blotting device where blotting is performed. In the aforementioned process, it takes at least 15 to 25 minutes simply to take out the gel, transfer the gel between different platforms, and mount the gel in place. Should any accident occur during the process, it is very likely that the electrophoresis and blotting will be affected or even fail. The complicated operation steps not only are time-consuming, but also require the operator to be highly skilled and experienced in order to complete each step successfully.

Therefore, the conventional western blot method has certain limitations in efficiency and reliability, which urgently demands improved and simplified solutions.

### BRIEF SUMMARY OF THE INVENTION

In view of the problems stated above, the inventor of the present invention developed a novel analysis platform by improving and integrating the three independent pieces of apparatus required for gel casting, electrophoresis, and blotting. The resulting platform not only can save time, effort, and hardware cost, but also performs well in protein analysis.

In one aspect, the present invention provides a protein analysis platform, comprising: a platform body comprising: a gel working unit provided in a top portion of the platform body and comprising a gel accommodation area for accommodating at least one gel; at least one electrophoresis tank provided along a side of the gel accommodation area and provided with at least one electrode; and a blotting layer stack provided in a bottom portion of the gel working unit and comprising an electrode layer, wherein a removable bottom plate is provided between the gel working unit and the blotting layer stack and detachably corresponds to a bottom side of the gel accommodation area.

In some embodiments, the gel working unit further comprises an upper cover plate, wherein the upper cover plate is detachably provided above the gel accommodation area.

In some embodiments, the gel working unit further comprises at least one upper cover plate locking element, wherein the upper cover plate locking element is provided on at least one side of the gel working unit to press the upper cover plate toward the gel accommodation area.

In some embodiments, the gel working unit further comprises a lateral baffle plate, wherein the lateral baffle plate is detachably provided between the gel accommodation area and the electrophoresis tank.

In some embodiments, the lateral baffle plate comprises an elastic layer provided in such a way that the elastic layer faces the gel accommodation area, wherein the elastic layer comprises rubber, plastic, or silicone.

In some embodiments, the gel working unit further comprises at least one lateral baffle plate locking element, wherein the lateral baffle plate locking element is provided on at least one side of the gel working unit configured to press the lateral baffle plate toward the gel accommodation area.

In some embodiments, the blotting layer stack further comprises a filter paper layer and a sponge layer, wherein the filter paper layer is provided above the electrode layer, and wherein the sponge layer is provided between the filter paper layer and the electrode layer.

In some embodiments, the platform body further comprises a second blotting layer stack provided above the gel working unit, wherein the second blotting layer stack comprises a second electrode layer and a polymer membrane layer, and wherein the second electrode layer is provided above the polymer membrane layer.

In some embodiments, the polymer membrane layer comprises polyvinylidene difluoride (PVDF).

In some embodiments, the second blotting layer stack further comprises a second filter paper layer and a second sponge layer, wherein the second filter paper layer is provided between the second electrode layer and the polymer membrane layer, and wherein the second sponge layer is provided between the second filter paper layer and the second electrode layer.

In some embodiments, the second blotting layer stack further comprises a blotting layer stack locking element provided above the second electrode layer and configured to be pressed toward the gel accommodation area.

In some embodiments, the electrode layer and the second electrode layer comprise platinum or titanium.

In some embodiments, the removable bottom plate is a rubber plate, a plastic plate, a glass plate, a ceramic plate, or a ceramic composite substrate.

In some embodiments, the removable bottom plate is the ceramic composite substrate, wherein the ceramic composite substrate comprises a ceramic substrate and a film, wherein the film is provided on an upper surface of the ceramic substrate, and wherein the ceramic substrate comprises alumina or aluminum nitride.

In some embodiments, the film comprises polytetrafluoroethylene (PTFE).

In some embodiments, the platform body has a lateral side further provided with a removable bottom plate accommodation groove for accommodating the removable bottom plate.

In some embodiments, the platform body further comprises a heat dissipation bottom plate provided under the blotting layer stack.

In some embodiments, the heat dissipating bottom plate is an alumina ceramic bottom plate, an aluminum nitride ceramic bottom plate, a thermoelectric cooling chip, or cooling fins.

In some embodiments, the platform body is peripherally provided with at least one supporting pillar for elevating the heat dissipating bottom plate.

In another aspect, the present invention also provides a use of the protein analysis platform mentioned above, wherein the protein analysis platform is used for western blot method or a next-generation western blot method.

The present invention is advantageous over the prior art in that gel casting, electrophoresis, and blotting can be carried out on a single analysis platform without moving the gel, installing additional apparatus, or changing apparatus, thereby saving the cost for the three discontinuous steps and separate apparatus in the conventional method; and that the protein analysis platform of the present invention works as well as, or even better than commercially available apparatus in individual steps. Thus, the present invention provides an analysis platform that features in high testing performance, rapidity, consistency, and high reliability. The present invention also provides a use of the protein analysis platform in biochemical testing.

### BRIEF DESCRIPTION OF THE SEVERAL VIEWS OF THE DRAWINGS

In order to make the above and other objects, features, advantages and embodiments of the present invention more obvious and understandable, the drawings are described as follows:

FIG. 1 to FIG. 4 illustrate structural diagrams of the platform body in an embodiment;

FIG. 5 illustrates photographs showing the electrophoresis results in an embodiment;

FIG. 6 illustrates photographs of the PVDF membranes in an embodiment;

FIG. 7 illustrates a photograph showing the electrophoresis result in an embodiment;

FIG. 8 illustrates a photograph showing the electrophoresis result in an embodiment;

FIG. 9 illustrates a photograph of the PVDF membrane in an embodiment;

FIG. 10 illustrates a comparison of gel casting and electrophoresis performances in an embodiment;

FIG. 11 illustrates the linear fitting equations in an embodiment, wherein the equations are used to determine the molecular weights of unknown proteins;

FIG. 12 illustrates a comparison of blotting performances in an embodiment;

FIG. 13 illustrates the electrophoresis results in an embodiment, demonstrating the effects of heat dissipation bottom plates of different materials on electrophoresis bands; and

FIG. 14 illustrates a bar chart showing the overall operation time of the protein analysis platform of the present invention in comparison with conventional devices.

### DETAILED DESCRIPTION OF THE INVENTION

The present invention is expounded below by describing some modes of implementation of the invention with reference to the accompanying drawings. In the drawings, identical elements are indicated by the same reference numeral, and the size or thickness of each element may be exaggerated for the sake of clarity.

As used herein, the term “or” means “and/or” unless otherwise stated. As used herein, the term “comprise or include” does not exclude the existence of one or more components, steps, operations, and/or elements in addition to the stated one(s) or exclude the addition of one or more components, steps, operations, and/or elements to the stated one(s). As used herein, the terms “comprise,” “include,” “contain,” “encompass,” and “have” are interchangeable without limitation. The indefinite article “a” connotes that the number of the syntactic object of the article is one or more than one (i.e., at least one).

In some embodiments, one of the objectives of the present invention is achieved by providing a protein analysis platform. The protein analysis platform includes a platform body. The platform body includes: a gel working unit provided in a top portion of the platform body and comprising a gel accommodation area for accommodating at least one gel; at least one electrophoresis tank provided along one side of the gel accommodation area and provided with at least one electrode; and a blotting layer stack provided in a bottom portion of the gel working unit and including an electrode layer, wherein a removable bottom plate is provided between the gel working unit and the blotting layer stack and detachably corresponds to the bottom side of the gel accommodation area.

In some embodiments, the gel working unit further includes an upper cover plate, wherein the upper cover plate is detachably provided above the gel accommodation area.

In some embodiments, the gel working unit further includes at least one upper cover plate locking element, wherein the upper cover plate locking element is provided on at least one side of the gel working unit to press the upper cover plate toward the gel accommodation area. In particular, the upper cover plate can provide a gel with a flat upper surface and isolate the gel from air to effectively make it easier for the gel to take shape.

More specifically, the upper cover plate locking element includes, but not limited to, a screw, a clamp, a cam lock, an elastic strap, or a combination thereof. Preferably, the upper cover plate locking element is a cam lock, which can effectively increase the adaptability of the upper cover plate locking element to a power source for automation, such as a servo motor.

In some embodiments, the gel working unit further includes a lateral baffle plate, and the lateral baffle plate is detachably provided between the gel accommodation area and the electrophoresis tank.

In some embodiments, the lateral baffle plate includes an elastic layer, wherein the elastic layer is provided in such a way that it faces the gel accommodation area, and wherein the elastic layer includes rubber, plastic, or silicone to provide enhanced airtightness when the elastic layer is compressed, and to prevent the gel from leaking after the gel casting step.

In some embodiments, the gel working unit further includes at least one lateral baffle plate locking element, and the lateral baffle plate locking element is provided on at least one side of the gel working unit to press the lateral baffle plate toward the gel accommodation area.

More specifically, the lateral baffle plate locking element includes, but is not limited to, a screw, a clamp, a cam lock, an elastic strap, or a combination thereof. Preferably, the lateral baffle plate locking element is a cam lock, which can effectively increase the adaptability of the lateral baffle plate locking element to a power source for automation, such as a servo motor.

In some embodiments, the blotting layer stack further includes a filter paper layer and a sponge layer. The filter paper layer is provided above the electrode layer, and the sponge layer is provided between the filter paper layer and the electrode layer.

In some embodiments, the platform body further includes a second blotting layer stack provided above the gel working unit, wherein the second blotting layer stack includes a second electrode layer and a polymer membrane layer, and wherein the second electrode layer is provided above the polymer membrane layer.

In some embodiments, the polymer membrane layer includes nitrocellulose (NC) or polyvinylidene difluoride (PVDF). Preferably, the polymer membrane layer includes PVDF.

In some embodiments, the second blotting layer stack further includes a second filter paper layer and a second sponge layer. The second filter paper layer is provided between the second electrode layer and the polymer membrane layer, and the second sponge layer is provided between the second filter paper layer and the second electrode layer.

In some embodiments, the second blotting layer stack further includes a blotting layer stack locking element provided above the second electrode layer and configured to be pressed toward the gel accommodation area.

More specifically, the blotting layer stack locking element includes, but is not limited to, a screw, a clamp, a cam lock, an elastic strap, or a combination thereof. Preferably, the blotting layer stack locking element is a cam lock, which can effectively increase the adaptability of the blotting layer stack locking element to a power source for automation, such as a servo motor.

In some embodiments, the materials of electrode layer and second electrode layer include platinum or titanium. In some other embodiments, the electrode layer and the second electrode layer are platinum threads, platinum plates, or platinum-plated titanium plates. In some preferred embodiments, the materials of electrode layer and second electrode layer include platinum-plated titanium. Without being bound by any particular theory, using the preferred material is effective in enhancing the uniformity of blotting.

In some embodiments, the removable bottom plate is a rubber plate, a plastic plate, a glass plate, a ceramic plate, or a ceramic composite substrate.

In some embodiments, the removable bottom plate is a ceramic composite substrate, which includes a ceramic substrate and a film. Wherein the film is provided on the upper surface of the ceramic substrate, and wherein the ceramic substrate includes alumina or aluminum nitride. In some embodiments, the ceramic substrate preferably includes aluminum nitride. The inventor of the present invention has found that an aluminum nitride ceramic bottom plate has a relatively high heat transfer coefficient; therefore, an aluminum nitride ceramic bottom plate is used in the invention to effectively increase heat dissipation efficiency. In the electrophoresis step, the aluminum nitride ceramic bottom plate is provided under the gel to dissipate heat, to improve the accumulation of heat during electrophoresis, thereby allowing the electrophoresis bands separated by electrophoresis to be straight.

In some embodiments, the film includes polytetrafluoroethylene (PTFE), which is commonly known as Teflon. Based on the hydrophobicity and relatively low adhesion force of Teflon, attaching a Teflon film to the ceramic substrate or coating the ceramic substrate with a Teflon film helps reduce the adhesion between the electrophoresis gel and the ceramic substrate, so that the ceramic substrate can be easily pulled out, i.e., removed, by the user without the gel being pulled out along with or deformed by the ceramic substrate.

Besides, when the structure of the protein analysis platform of the present invention is considered as a whole, the use of the removable bottom plate helps to increase the structural strength of the platform. The removable bottom plate also facilitates user operation.

In some embodiments, a lateral side of the platform body is provided with a removable bottom plate accommodation tank (or slidable bottom plate accommodation tank) for accommodating the removable bottom plate (or slidable bottom plate).

In some embodiments, the platform body further includes a heat dissipation bottom plate provided under the blotting layer stack. More specifically, the heat dissipation bottom plate is an alumina ceramic bottom plate, an aluminum nitride ceramic bottom plate, a thermoelectric cooling chip, or cooling fins.

In some embodiments, the platform body is peripherally provided with at least one supporting pillar for elevating the heat dissipation bottom plate. More specifically, the number of the supporting pillar provided along the periphery of the platform body is two, three, or four; the specific number can be adjusted by a person of ordinary skill in the art according to the purpose or requirement of elevation and is not subject to any particular limitation as far as the present invention is concerned.

In some embodiments, another objective of the present invention is to provide a use of the aforementioned protein analysis platform in a northern blot method, a southern blot method, a western blot method, or a next-generation western blot method. Preferably, the use of the aforementioned protein analysis platform is in a western blot method or a next-generation western blot method.

In some embodiments, another objective of the present invention is to provide an automated analysis module including the aforementioned analysis platform. The automated analysis module is configured to identify the current experimental/working status and thereby control the operation of the analysis platform automatically.

In some embodiments, samples suitable for use with the analysis platform include, but are not limited to, proteins, ribonucleic acids (RNAs), and deoxyribonucleic acids (DNAs).

In some embodiments, the method of producing the platform body includes rubber or plastic processing, a three-dimensional (3D) printing technique such as stereolithography (SLA), or fused deposition modeling (FDM). The producing method to be used can be adjusted by a person of ordinary skill in the art according to the purpose or requirements of producing the platform body and is not subject to any limitation as far as the present invention is concerned.

In some embodiments, the gel formed by the gel casting step includes an upper gel (stacking gel) and a lower gel (separation gel), wherein the lower gel is on the side close to the lateral baffle plate. More specifically, the upper gel includes a plurality of loading wells intended to be loaded with samples that are to go through the subsequent electrophoresis step. The dimensions of the loading wells include, but not limited to, 5 (length)×3 (width)×1 (depth) mm or 3.5 (length)×4 (width)×1 (depth) mm. In some preferred embodiments, the dimensions of the loading wells are 5 (length)×3 (width) ×1 (depth) mm. The inventor of the present invention has found that a better compression effect can be achieved with wider loading wells. Moreover, in some embodiments, an increase in the transverse dimension of the loading wells helps increase the sharpness of the electrophoresis bands if the quantity of the loaded samples is relatively great.

In some embodiments, with a view to reducing the effect of the heat of the electrophoresis process on the electrophoresis bands, the gel can be designed, in areas corresponding to the loading wells, in such a way that all the bands will be as far away from the two lateral sides of the gel accommodation area as possible. This structure design allows the bands to have relatively uniform separation temperatures and separate at relatively uniform speeds. For instance, the gel accommodation area can be enlarged on each of the two lateral (i.e., left and right) sides by, for example, but not limited to, 1 mm, 2 mm, 2.1 mm, 2.2 mm, 2.3 mm, 2.4 mm, 2.5 mm, 2.6 mm, 2.7 mm, 2.8 mm, 2.9 mm, or 3 mm.

In some embodiments, the gel casting method used in the present invention includes horizontal gel casting or vertical gel casting, preferably vertical gel casting.

In some embodiments, the power source to which the protein analysis platform is connected during the electrophoresis step has a constant voltage in the range from 150 V to 450 V, such as 150 V, 160 V, 170 V, 180 V, 190 V, 200 V, 250 V, 300 V, 350 V, 400 V, or 450V, preferably in the range from 250 V to 300 V. In the absence of any limitations imposed by a specific theory, the inventor of the present invention has found that when the constant voltage is 300 V, the band positions in an electrophoresis result almost coincide with the peak intensity values obtained by quantification, and the standard deviation is also desirable (the result not shown).

In some embodiments, the heat dissipation bottom plate is provided with a plurality of holes, and the holes include elongated holes or circular holes. The heat dissipation bottom plate has a porosity in the range from 0.190% to 0.480%, such as, but not limited to, 0.190%, 0.191%, 0.192%, 0.193%, 0.194%, 0.195%, 0.196%, 0.197%, 0.198%, 0.199%, 0.200%, 0.300%, 0.400%, 0.410%, 0.420%, 0.430%, 0.440%, 0.450%, 0.460%, 0.470%, 0.471%, 0.472%, 0.473%, 0.474%, 0.475%, 0.476%, 0.477%, 0.478%, 0.479%, or 0.480%.

More specifically, when the heat dissipation bottom plate is placed under the gel, the holes in the heat dissipation bottom plate help to dissipate heat during the electrophoresis process. Moreover, when the heat dissipation bottom plate is placed under the gel, the holes in the heat dissipating bottom plate help improve blotting efficiency during the blotting process.

In some embodiments, the blotting step is such that the smaller the distance between the electrode layer and the second electrode layer, the better the blotting effect. More specifically, given a constant current of 250 mA and an operation time of 10 minutes, the distance is in the range from 11 mm to 14 mm, such as, but not limited to, 11 mm, 11.5 mm, 11.51 mm, 11.52 mm, 11.53 mm, 11.54 mm, 11.55 mm, 11.56 mm, 11.57 mm, 11.58 mm, 11.59 mm, 11.60 mm, 11.70 mm, 11.80 mm, 11.90 mm, 12.00 mm, 12.5 mm, 13.00 mm, 13.5 mm, 13.51 mm, 13.52 mm, 13.53 mm, 13.54 mm, 13.55 mm, 13.56 mm, 13.57 mm, 13.57 mm, 13.59 mm, 13.60 mm, 13.70 mm, 13.80 mm, 13.90 mm, or 14.00 mm, preferably in the range from 11.55 mm to 13.55 mm. Besides, given a constant current of 250 mA to 600 mA and an operation time of 6 to 10 minutes, the distance between electrode layers is in the range from 8 mm to 11 mm, such as, but not limited to, 8 mm, 8.1 mm, 8.2 mm, 8.3 mm, 8.4 mm, 8.5 mm, 8.51 mm, 8.52 mm, 8.53 mm, 8.54 mm, 8.55 mm, 8.56 mm, 8.57 mm, 8.58 mm, 8.59 mm, 8.6 mm, 8.7 mm, 8.8 mm, 8.9 mm, 9.0 mm, 9.5 mm, 10.0 mm, 10.5 mm, 10.51 mm, 10.52 mm, 10.53 mm, 10.54 mm, 10.55 mm, 10.56 mm, 10.57 mm, 10.58 mm, 10.59 mm or 11.0 mm, preferably in the range from 8.55 mm to 10.55 mm.

### EMBODIMENTS

### Embodiment 1

### Preparation of the Platform Body

Method: Software SOLIDWORKS was used to make a 3D diagram of a platform body 10, and the diagram was converted into engineering drawings in order to review the dimensions of the platform body 10. Next, the graphic files were converted into the STL format by an SLA 3D printing technique and output to an SLA printer. The SLA printer irradiated photosensitive resin with laser light of specific energy to solidify and thereby shape the resin. After that, the object obtained by 3D printing went through a fixing process in which the SLA printed object was fixedly connected to the rubber, plastic or silicone foam elements with AB glue (i.e., a two-component adhesive) to obtain the platform body 10.

### Embodiment 2

### Component Arrangement of the Protein Analysis Platform

Two platform configurations are described below by way of example.

### [Configuration 1]

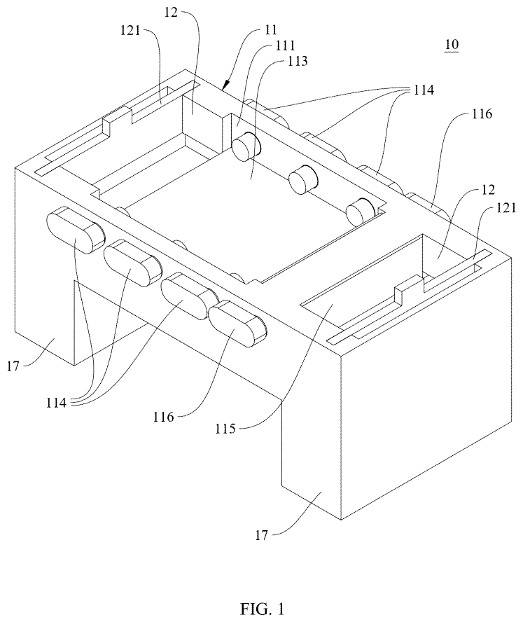

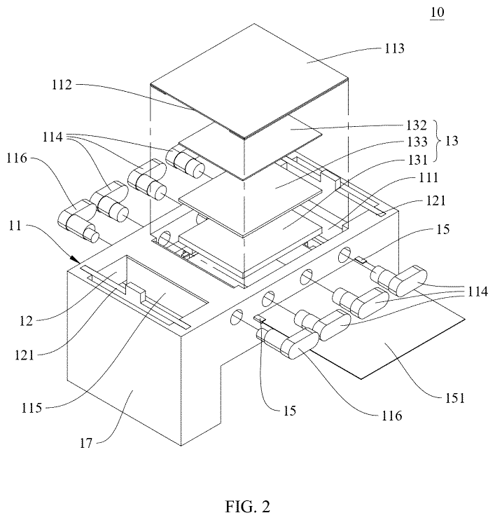

Referring to FIG. 1 and FIG. 2, the analysis platform includes a platform body 10, which includes: a gel working unit 11 provided in a top portion of the platform body 10, wherein the gel working unit 11 includes a gel accommodation area 111 for accommodating a gel 112; two electrophoresis tanks 12 each provided along one of two opposite sides of the gel accommodation area 111 and each provided with an electrode 121; and a blotting layer stack 13 provided in a bottom portion of the gel working unit 11 and including an electrode layer 131 (which includes platinum). In addition, a removable bottom plate 151 is detachably provided between the gel working unit 11 and the blotting layer stack 13 and corresponds to the bottom side of the gel accommodation area 111.

The gel working unit 11 further includes an upper cover plate 113. The upper cover plate 113 is detachably provided above the gel accommodation area 111.

The gel working unit 11 further includes six upper cover plate locking elements 114. The upper cover plate locking elements 114 are provided on two opposite sides of the gel working unit 11 to press the upper cover plate 113 toward the gel accommodation area 111 or to press the gel accommodation area 111 toward the upper cover plate 113. Each of the upper cover plate locking elements 114 is a cam lock.

The gel working unit 11 further includes a lateral baffle plate 115. The lateral baffle plate 115 is provided between the gel accommodation area 111 and one of the electrophoresis tanks 12 in a removable manner. The lateral baffle plate 115 includes an elastic layer facing the gel accommodation area 111, and the elastic layer includes silicone foam.

The gel working unit 11 further includes two lateral baffle plate locking elements 116. The two lateral baffle plate locking elements 116 are provided on two opposite sides of the gel working unit 11 to press the lateral baffle plate 115 toward the gel accommodation area 111. Each of the lateral baffle plate locking elements 116 is a cam lock.

The blotting layer stack 13 further includes a filter paper layer 132 and a sponge layer 133. The filter paper layer 132 is provided above the electrode layer 131, and the sponge layer 133 is provided between the filter paper layer 132 and the electrode layer 131.

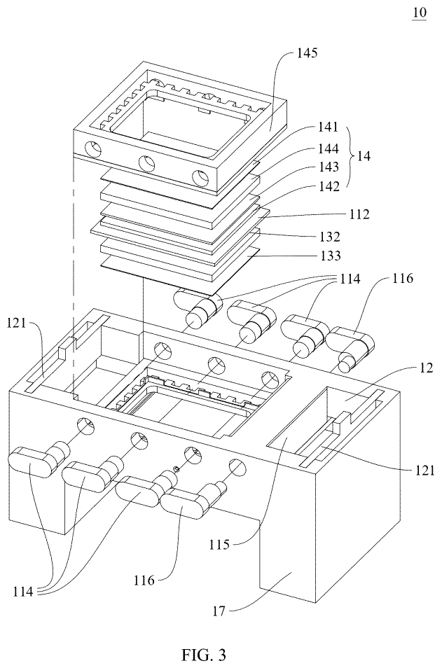

Referring to FIG. 3, the platform body 10 further includes a second blotting layer stack 14 provided above the gel working unit 11, and the second blotting layer stack 14 includes a second electrode layer 141 (which includes platinum) and a PVDF membrane (i.e., a polymer membrane layer) 142, wherein the second electrode layer 141 is provided above the polymer membrane layer 142. The PVDF membrane is chosen for use as the polymer membrane layer 142 because protein samples are used in the following embodiments by way of example.

The second blotting layer stack 14 further includes a second filter paper layer 143 and a second sponge layer 144. The second filter paper layer 143 is provided between the second electrode layer 141 and the polymer membrane layer 142, and the second sponge layer 144 is provided between the second filter paper layer 143 and the second electrode layer 141.

The second blotting layer stack 14 further includes a blotting layer stack locking element 145 provided above the second electrode layer 141 and configured to be pressed toward the gel accommodation area 111.

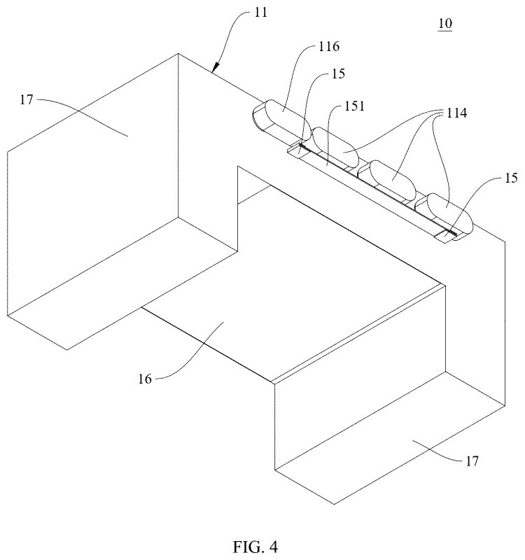

Referring to FIG. 4, a lateral side of the platform body 10 is provided with a removable bottom plate accommodation tank 15 for accommodating the removable bottom plate 151. The removable bottom plate 151 is an aluminum nitride ceramic substrate whose upper surface of the removable bottom plate 151 is provided with a film containing PTFE.

The platform body 10 further includes a heat dissipation bottom plate 16 provided under the blotting layer stack 13. The heat dissipation bottom plate 16 is an aluminum nitride ceramic substrate.

The four sides of the platform body 10 are each provided with a supporting pillar 17 to elevate the heat dissipation bottom plate 16.

### [Configuration 2]

Configuration 2 of the protein analysis platform is structurally different from configuration 1 in that the heat dissipation bottom plate 16 is a panel-shaped structure provided under the gel working unit 11, and that the heat dissipation bottom plate 16 is an alumina ceramic substrate formed with a plurality of holes. These holes must be closed during electrophoresis in order to prevent the casting gel from leaking.

Another difference is that, when electrophoresis is completed, configuration 2 requires the holes in the bottom of the heat dissipation bottom plate 16 to be opened and exposed, thereby allowing contact between the gel 112 and the blotting buffer, which enhances blotting efficiency. Besides, configuration 2 requires the entire analysis platform to be placed in a blotting tank, and the blotting tank to be filled with the blotting buffer until the analysis platform is immersed.

### Embodiment 3

### Process Flow of The Gel Casting, Electrophoresis, and Blotting of the Experimental Group

The process flows corresponding to the two analysis platform configurations are described below with reference to FIG. 1 to FIG. 4 but without being restrictive of the present invention.

### [Process Flow Corresponding to Configuration 1]

### 1. Gel Casting

First, putting the removable bottom plate 151 into the removable bottom plate accommodation tank 15.

Next, based on the principles of vertical gel casting, the upper cover plate 113 and the lateral baffle plate 115 were put into the platform body 10, i.e., with the upper cover plate 113 placed above the gel accommodation area 111 in a removable manner, and the lateral baffle plate 115 put between the gel accommodation area 111 and electrophoresis tanks 12 in a removable manner. Once the lateral baffle plate 115 (together with the silicone foam thereof) is put into the analysis platform, the cam locks 116 were inserted in place and rotated upward in order to press the lateral baffle plate 115 and the silicone foam upward to the required level of tightness.

The analysis platform was then placed vertically, and the lower-gel solution was introduced into the platform. After that, a small amount of isopropyl alcohol was introduced into the platform to flat the upper edge of the lower gel. After the lower gel took shape, the residual isopropyl alcohol was poured out. The analysis platform was subsequently placed in the horizontal position. After inserting the comb, the upper-gel solution was introduced into the platform and allowed to take shape.

Once the upper gel was formed, the cam locks 116 were rotated to allow removal of the lateral baffle plate 115 so that the gel 112 could contact the electrophoresis buffer, meaning the following electrophoresis step could be performed in succession without having to move the gel 112.

### 2. Electrophoresis

Pre-stained protein markers serving as samples were injected into the loading wells. Then, electrodes 121 were placed in the electrophoresis tanks 12 on both sides and were connected to the power supply. Introducing the electrophoresis buffer into the platform, and then start the electrophoresis step.

When electrophoresis was completed, the upper cover plate 113 was removed, and the blotting step continuously performed under the condition that the gel 112 remains in its original position.

### 3. Blotting

After completion of the electrophoresis step, the removable bottom plate 151 was pulled out carefully, and the second blotting layer stack 14 (i.e., the upper half portion of the blotting sandwich) was placed above the gel 112. The middle area of the analysis platform was then filled with the blotting buffer until the second blotting layer stack 14 was immersed. Blotting began as soon as electricity was supplied.

It is worth mentioning that when the removable bottom plate 151 was pulled out, the bottom of the gel 112 was exposed and contacted to the blotting layer stack 13, wherein the blotting layer stack 13 was placed under the removable bottom plate 151 in advance. Wherein, the blotting layer stack 13 included, sequentially in a top-to-bottom direction, the filter paper layer 132, the sponge layer 133, and the electrode layer 131. After the removable bottom plate 151 was pulled out, the sponge layer 133 expanded and thus generated a pushing force toward the gel 112; as a result, the filter paper layer 132 was pushed upward and made direct contact with the gel 112. The aforementioned design reduces the interference/operations required between the electrophoresis step and the blotting step, including, for example, the difficult operation of putting the filter paper layer 132 and the sponge layer 133 under the gel 112, and prevents the electrical equipment from prolonged exposure to the corrosive electrolytic solution.

Moreover, that the blotting layer stack 13 was placed under the removable bottom plate 151 before the gel casting step allowed the blotting step to be started smoothly. This technical feature greatly increases the continuity and efficiency of the operations, shortens the time required for the operations, and reduces the effects of variables on batch operation.

### Results

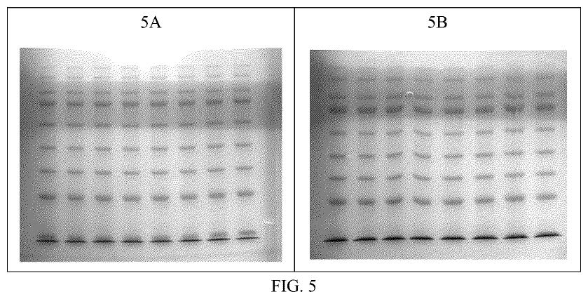

FIG. 5 shows photographs of the gels having completed the electrophoresis step. The electrophoresis result in FIG. 5A was photographed under the following electrophoresis conditions: a constant voltage of 250 V, a current set at 69-81 mA, and a run time of 29 minutes. The electrophoresis result in FIG. 5B was photographed under the following electrophoresis conditions: a constant voltage of 300 V, a current set at 79-91 mA, and a run time of 22 minutes.

The results show that the bands corresponding to the pre-stained protein markers, which have different molecular weights, were clearly separated.

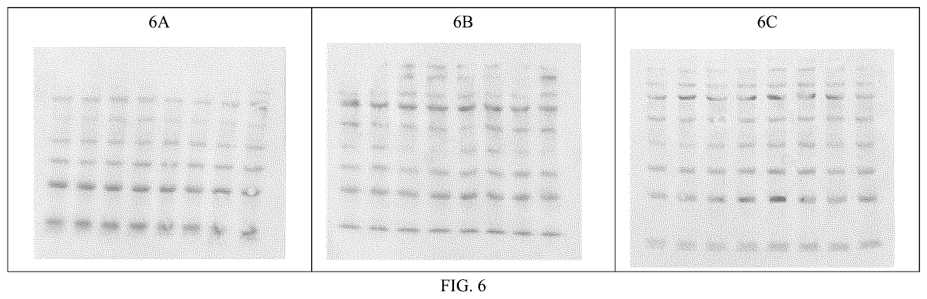

FIG. 6 shows photographs of the PVDF membranes having completed the blotting step. More particularly, FIG. 6 shows a comparison of the blotting results corresponding to an electrode plate-to-electrode plate distance (i.e., the distance between the electrode layer 131 and the second electrode layer 141) of 8.55 mm and a blotting time of 6 minutes. The blotting result in FIG. 6A was photographed under such blotting conditions as a constant current of 350 mA and a power of 6650-11900 W. The blotting result in FIG. 6B was photographed under such blotting conditions as a constant current of 500 mA and a power of 14000-28000 W. The blotting result in FIG. 6C was photographed under such blotting conditions as a constant current of 600 mA and a power of 42000-46800 W.

The inventor of the present invention has found that the smaller the distance between the second electrode layer 141 and the electrode layer 131, the better the blotting effect obtaining. Wherein, this phenomenon was attributable to the fact that in the analysis platform used in this embodiment, the blotting sandwich (composed of the blotting layer stack 13 and the second blotting layer stack 14) uses the second electrode layer 141 and the electrode layer 131 to clamp the sponge layer 133, the filter paper layer 132, the PVDF membrane and the gel 112. The smaller the distance between the second electrode layer 141 and the electrode layer 131, the better the clamping effect, thereby enhancing blotting effect. More specifically, when the electrode plate-to-electrode plate distance was reduced to 8.55 mm, and the current was raised to 600 mA, molecules not larger than 93 kDa were effectively blotted on the PVDF membrane, and the molecular weight of the largest molecules that could be blotted was 125 kDa.

### [Process Flow Corresponding to Configuration 2]

### 1. Gel Casting

Based on the principles of horizontal gel casting, the platform body 10 was placed in the horizontal position, and two lateral baffle plates 115 (each divided into an upper baffle plate and a lower baffle plate, not indicated in the drawings) were each placed between the gel accommodation area 111 and the electrophoresis tanks 12 in a removable manner. Then, the prepared lower-gel solution was introduced into the platform, and the upper cover plate 113 was put in place after the introduction of the lower-gel solution was completed.

Once the lower gel took shape, the upper baffle plates 115 were removed and put at the corresponding highest positions of the gel shaping area to prevent the upper-gel solution from leaking during gel casting process. The prepared upper-gel solution was then introduced into the platform, and the upper cover plate 113 was put in place. The comb was subsequently inserted into the upper-gel solution.

After the upper gel took shape, the comb was removed, and then the upper baffle plates 115 and the lower baffle plates 115 were removed to allow subsequent contact between the gel 112 and the electrophoresis buffer, thereby allowing the electrophoresis step to be carried out in succession without having to move the gel 112.

### 2. Electrophoresis

Pre-stained protein markers serving as samples were injected into the loading wells.

Then, placing electrodes 121 on both sides of electrophoresis tanks 12 and were connected to the power supply, introducing the electrophoresis buffer into the platform, and then start the electrophoresis step.

When electrophoresis was completed, the upper cover plate 113 was removed, and the blotting step continuously performed under condition of the gel 112 remaining at original position.

### 3. Blotting

Placing the blotting layer stack, which included, sequentially in a top-to-bottom direction, the second electrode layer 141, the second sponge layer 144, the second filter paper layer 143, the polymer membrane layer 142 (PVDF membrane), the gel 112, and the electrode layer 131.

The entire analysis platform was put into a blotting tank, which was then filled with the blotting buffer until the analysis platform was immersed. Blotting began as soon as electricity was supplied.

### Results

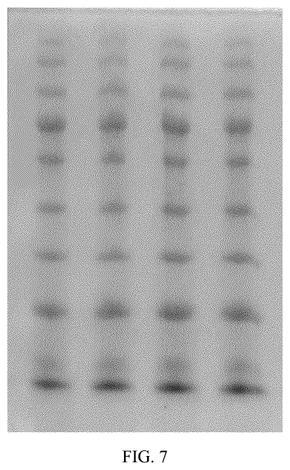

FIG. 7 shows an electrophoresis gel photographed under the following electrophoresis conditions: a constant voltage of 200 V was used, the protein marker bands reached a 4.5-cm separation distance within 62 minutes, and a cooling environment was provided around the analysis platform (e.g., by way of water cooling blocks in which 2° C. water was circulated). It can be seen in the photograph that the bands of the protein markers, which have different molecular weights, were clearly separated in the electrophoresis gel. Moreover, as the analysis platform was provided with the heat dissipation bottom plate 16 (which was an alumina ceramic substrate), it can be seen in the photograph that the protein marker bands were improved in shape: the bands are straight separated bands rather than bands showing a smiling curve.

### Embodiment 4

### Process Flow of the Gel Casting, Electrophoresis, and Blotting Operations of the Control Group

### Process Flow

The following steps were carried out using commercially available gel casting, electrophoresis, and blotting devices, and the conventional western blot method was employed in the experiment. The experimental results are used herein as the control-group results.

Gel casting step: With reference to the user manual of Dual Gel Caster (Hoefer™) (see Table 1), a gel sandwich was assembled and secured in the corresponding clamp assembly. Distilled water was added into the gel sandwich to make sure that no water leaked out. The water was poured out, and filter paper was used to absorb the remaining water in the gel sandwich until the gel sandwich was dry. The clamp assembly and the gel sandwich were then placed in the casting cradle. Using a pipette, the prepared lower-gel solution was introduced into the gel sandwich to a predetermined height, before a small amount of isopropyl alcohol was added to isolate the lower-gel solution from air, thereby accelerating gel formation, and to flat the upper edge of the lower gel. Once the lower gel solidified, the residual isopropyl alcohol was poured out, and the upper gel accommodation area was rinsed with clean water and then dried with paper towel. Following that, the upper-gel solution was added, and the comb was inserted into the upper-gel solution, which was allowed to rest for 15 to 20 minutes until the gel took shape. The resulting gel was removed from the casting cradle and roughly washed in order to be used in the following electrophoresis analysis of proteins.

Electrophoresis step: The comb was carefully removed from the solidified gel, and the screws on the clamp assembly were loosened. After that, the glass plate, the spacers, the ceramic plate, and the solidified gel were moved out and put into an electrophoresis tank, wherein the Hoefer SE250 electrophoresis platform (see Table 1) was used to run electrophoresis of proteins. The electrophoresis buffer was slowly poured into the electrophoresis tank, and the loading wells in the upper gel were loaded with the pre-stained protein marker samples to be tested. After connecting the electrophoresis platform to the power supply, electrophoresis was performed under a constant voltage, and during the process, attention was paid to whether or not the target proteins moved out of the lower gel.

Blotting step: A vertical wet blotting platform (Hoefer TE22 Mighty Small Transfer Tank) was used. To begin with, the sponge, filter paper, and PVDF membrane were wetted with the blotting buffer. Then, the gel having completed the electrophoresis process was carefully detached from the glass and ceramic plates and put into the blotting sandwich formed between the blotting cassette panels, wherein the blotting sandwich includes, sequentially in a top-to-bottom direction, the blotting cassette upper panel, a piece of sponge, a piece of filter paper, the gel, the PVDF membrane, another piece of filter paper, another piece of sponge, and the blotting cassette lower panel. Following that, the blotting cassette was placed in a blotting tank, and the blotting tank was filled with the blotting buffer until full. Ice packs were put around the blotting platform for cooling purposes. The blotting platform was then connected to the power supply, and blotting took place under a constant current of 200 mA.

### Results

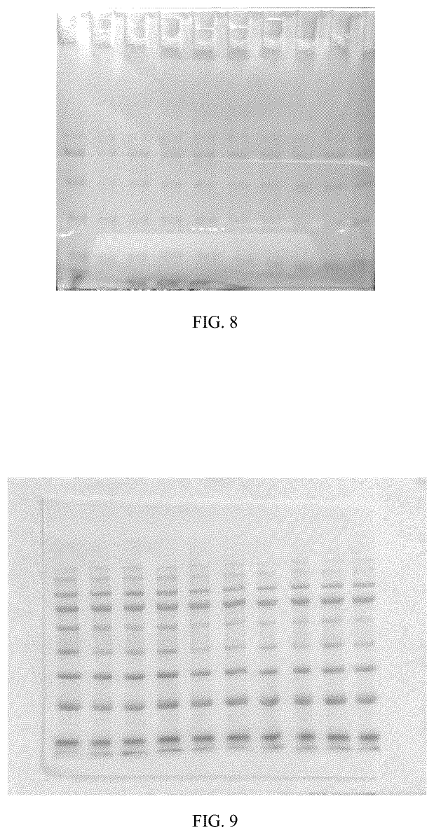

The electrophoresis result is shown in FIG. 8. An observation of sample migration in the upper gel reveals that, given a constant voltage of 90 V and a current in the range from 39 to 40 mA, compression to the upper edge of the separation gel took 15 minutes, and that when the constant voltage was subsequently raised to 120 V, separation occurred in the separation gel according to the molecular weights of the samples, during which process the current was about 61 mA, the time required to cause a 5-cm separation being 60 minutes. Thus, the total operation time of the electrophoresis is 75 minutes.

The blotting result is shown in FIG. 9. Given the use of the conventional blotting system and a constant current of 200 mA. the total blotting time is 120 minutes.

The instruments, consumables, and solution preparation methods used in the aforementioned experiments are listed in Table 1 to Table 5.

TABLE 1
Instruments used in the experiments

| Name of instrument | Description |
|---|---|
| Laser cutter | Commercially available product. Brand/manufacturer and |
| | model: GCC LaserPRO ™ Mercury II. For cutting objects to |
| | target sizes. Uses a laser tube that contains metal carbon |
| | dioxide. See Table 2 for operation parameters. |
| Gel casting platform | Commercially available gel casting platform. |
| (polyacrylamide gel | Brand/manufacturer and model: Dual Gel Caster (Hoefer ™, |
| platform) | Massachusetts, United States). |
| Electrophoresis platform | Commercially available vertical electrophoresis platform. |
| | Brand/manufacturer and model: Hoefer SE250. |
| Blotting platform | Commercially available blotting platforms, including a |
| | vertical wet blotting platform (Hoefer TE22 Mighty Small |
| | Transfer Tank) and a semi-dry blotting platform (Trans-Blot |
| | Turbo (Bio-Rad), California, United States). |
| Power supply | Commercially available product. Brand/manufacturer and |
| | model: GW Instek PSW 800-4.32. |
| Microbalance | Commercially available product. Brand/manufacturer and |
| | model: Mettler Toledo PB3002-S/FACT. |
| Pipette | Commercially available product. Brand/manufacturer and |
| | model: Sartorius Tacta ®. |
| Vortex mixer | Commercially available product. Brand/manufacturer and |
| | model: DLAB MX-F. This equipment can be set to operate |
| | at a constant speed of 500 rpm. |
| Constant-temperature | Commercially available product. Brand/manufacturer and |
| circulator | model: NESLAB RTE-110. This equipment can be used for |
| | constant-temperature heat dissipation during electrophoresis. |
| Photographic system | Commercially available product. Brand/manufacturer and |
| | model: Apple IPhone 14 Pro. Used for taking photographs |
| | and recording the gel casting, electrophoresis, and blotting |
| | results. |
| Image processing system | Java-based ImageJ Fiji software for quantitative analysis of |
| | the target area of an image. |

TABLE 2
Specifications of the laser cutter

| Model | Mercury II |
|---|---|
| Model Number | M-40V |
| Wavelength | 10.57-10.63 μm |
| Power | CO₂ 40 W |
| Input | 200-240 VAC, 50-60 Hz, max. 12 A |

TABLE 3
Consumables used in the experiments

| Name of consumable | Description |
|---|---|
| Gel reagent kit | Commercially available product. Brand/manufacturer: |
| | Bio-Rad. Model: TGX Stain-Free FastCast Acrylamide Kit, |
| | 10%. Requires the addition of TEMED |
| | (N,N,N′,N′-tetramethyl-ethane-1,2-diamine) and APS |
| | (ammonium persulfate). See the user manual of this |
| | consumable for the ratios for gel preparation. |
| Isopropyl alcohol | Commercially available product. Brand/manufacturer: |
| | Cheng Yi Chemical Co., Ltd. |
| Electrophoresis | Commercially available product. Brand/manufacturer: |
| concentrate | Bio-Rad. Specifications: 10× Tris/glycine/SDS buffer. |
| | Preparation example: 1 L of electrophoresis buffer is |
| | prepared by adding 100 mL of electrophoresis concentrate |
| | into 900 mL of distilled water; the resulting mixture is a 1× |
| | electrophoresis buffer and has a pH value of 8.3. |
| Blotting concentrate | Commercially available product. Brand/manufacturer: |
| | Bio-Rad. Specifications: 10× Tris/glycine buffer. |
| | Preparation example: 1 L of blotting buffer is prepared with |
| | 700 mL of distilled water + 100 mL of blotting concentrate + |
| | 200 mL of methanol; the resulting mixture is a 1× |
| | blotting buffer and has a pH value of 8.3. |
| PVDF membrane | Commercially available product. Brand/manufacturer: |
| | Merck. Model: Immobilon ®-E. Does not require |
| | impregnation with methanol. Hole diameter in membrane: |
| | 0.45 μm. |
| Pre-stained protein | Commercially available product. Brand/manufacturer: EBL |
| markers | Biotechnology Co., Ltd. Model: PPM-18010250. For |
| | visualizing protein transfer efficiency. The markers include |
| | ten protein bands, covering a range of molecular weights |
| | from 10 to 180 kDa. The fourth protein band from the top is |
| | coupled to a green dye, the eighth protein band from the top |
| | is coupled to a red dye, and the remaining protein bands are |
| | covalently coupled to a blue dye. |
| Methanol | Commercially available product. Brand/manufacturer: EBL |
| | Biotechnology Co., Ltd. For use in preparing the blotting |
| | buffer. |
| Filter paper for | Commercially available product. Brand/manufacturer: EBL |
| blotting | Biotechnology Co., Ltd. Made of 100% cotton fiber. |
| | Dimensions: 8 × 13.5 cm. Thickness: 1.3 mm. |
| Centrifugation tube | Commercially available product. Brand/manufacturer: |
| | Kirgen. For use in preparing polyacrylamide gel. |
| Platinum thread | Commercially available product. Brand/manufacturer: |
| | Lingo-Go Co., Ltd. Diameter: 0.3 mm. Platinum content: |
| | 99%. |
| Rubber/plastic plate | Commercially available products. Brand/manufacturer: |
| | Chiao Dar Acry & Advertisement Co., Ltd. Dimensions: 30 × |
| | 30 cm. Thickness: 1, 2, 3, 4, or 5 mm. Transparent plates |
| | are available. Rubber/plastic plates were cut to the required |
| | sizes by the laser cutter in order to make the machine used |
| | in the experiments. |
| Teflon film tape | Commercially available product. Brand/manufacturer: |
| | Sashay Technology Limited. Specifications: Teflon Tape |
| | S-0500, thickness 0.05 mm, width 110 mm. Resistant to |
| | strong acids and strong alkalis. Has a non-stick surface and |
| | an extremely low friction coefficient. Continuous |
| | application temperature: −73° C. to 260° C. This Teflon film |
| | tape has silicone on the back and can be directly adhered to |
| | an intended object, without having to apply additional |
| | adhesive; this helps reduce the tape thickness as much as |
| | possible. |
| Silicone foam | Commercially available product. Brand/manufacturer: |
| | Zheng-Yi Rubber Co., Ltd. Silicone foam features |
| | resistance to high temperature, satisfactory airtightness, |
| | resistance to water and oil, and electrical insulation; has a |
| | operating temperature of −20° C. to 320° C., a hardness of ±30A, |
| | and a density of 200-500 kg/m³; and was used in the |
| | experiments for leakage prevention and sealing purposes |
| | during the gel casting process. |
| Alumina ceramic and | Commercially available products. Brand/manufacturer: |
| aluminum nitride | Tensky International Co., Ltd. Dimensions: 114.5 × 114.5 |
| ceramic | mm. Thickness: 0.5 mm. See Table 4 for their properties at |
| | room temperature. |

TABLE 4
Properties of alumina ceramic and of aluminum nitride ceramic

| | | Aluminum nitride |
|---|---|---|
| Property | Alumina ceramic | ceramic |
| Density (g/cm³) | 3.6 | 3.32 |
| Water absorption (%) | 0 | 0 |
| Hardness (MPa) | 1440 | 1500 |
| Flexural strength (MPa) | 300 | 720 |
| Thermal conductivity (W/mK) | 20 | 170 |
| Max. operating | 1500 | 1700 |
| temperature (° C.) | | |
| Resistance to acidic/ | Strong | Strong |
| alkaline corrosion | | |

TABLE 5
Solution preparation methods used in the experiments

| Solution | Preparation method |
|---|---|
| Upper-gel solution | Component A (TAE-polyacrylamide) (1 mL) + |
| (polyacrylamide | component B (TBE-polyacrylamide) (1 mL) + |
| stacking gel) | TEMED (1 μL) + APS (10 μL) |
| Lower-gel solution | Component A (4 mL) + component B (4 mL) + |
| (polyacrylamide | TEMED (4 μL) + APS (40 μL) |
| separation gel) | |
| APS solution | APS (0.1 g) + distilled water (1 mL) |
| Electrophoresis buffer | Electrophoresis concentrate (0.1 mL) + |
| | distilled water (0.9 mL) |
| Wet blotting buffer | Wet blotting concentrate (0.1 mL) + methanol |
| | (0.2 mL) + distilled water (0.7 mL) |

### Embodiment 5

### Comparison of the Electrophoresis Results of the Improved Analysis Platform and of the Conventional Electrophoresis Platform

The improved analysis platform (of configuration 1 in embodiment 2) and the conventional gel casting platform and electrophoresis platform were analyzed using the same markers.

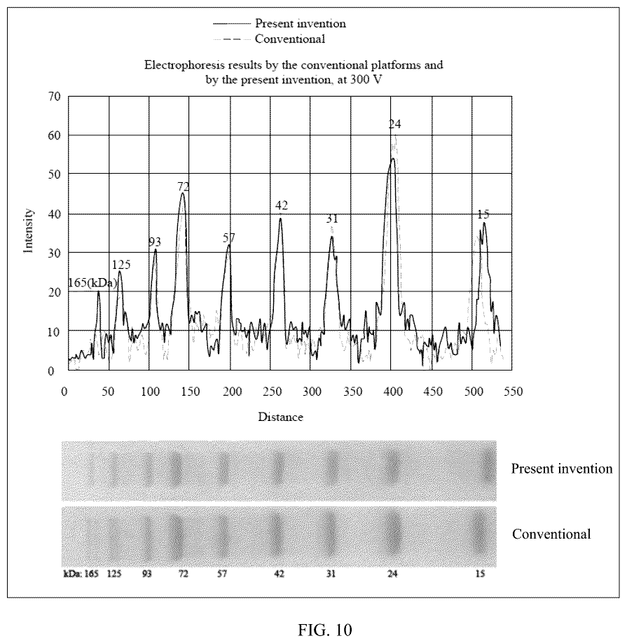

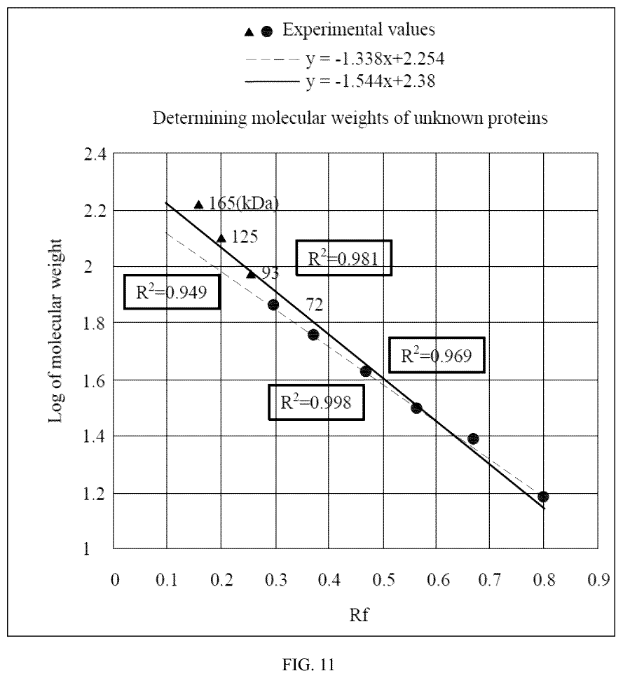

According to the analysis results in FIG. 10, all the markers, whose molecular weights are 15, 24, 31, 42, 57, 72, 93, 125, and 165 kDa, were successfully separated, including the marker with the lowest molecular weight (15 kDa), and each band has a distinct intensity peak after quantification. Therefore, it can be inferred that the improved analysis platform can produce the same/similar effects in the gel casting step and the electrophoresis step comparing to the commercially available gel casting platform and electrophoresis platform.

In addition, an integrated analysis of the molecular weights corresponding to eight of the bands and the migration distances of those bands under the suggested voltage of 300 V shows that in each lane, the migration distance of the band corresponding to the molecular weight of 15 kDa has the greatest standard deviation, i.e., 0.044, with the actual error being 0.1031 mm, and that the average standard deviation of the migration distances corresponding to the other molecular weights is 0.03346, which is also nominal and has no effect on the experimental results. It can be clearly seen that the marker bands in each lane of the electrophoresis result obtained from the improved analysis platform are practically aligned with their respective counterparts in the other lanes, wherein the marker bands in each lane of the electrophoresis are nearly perpendicular to the moving direction of the bands.

### Embodiment 6

### Verification of the Accuracy of the Gel Casting and Electrophoresis Results of the Analysis Platform

In this embodiment, the migration distances of the bands in the gel were used to estimate the molecular weight corresponding to each band.

### Method

An unknown protein can be identified by its relative migration distance in a gel electrophoresis test, i.e., by comparing the migration distance of the unknown protein with those of known proteins, or more particularly by comparing the molecular weights derived from the migration distances of the those proteins. This can be done by plotting a graph in which the Y axis represents the log of each protein's molecular weight while the X axis represents the relative migration distance (Rf), and by establishing a linear fitting equation accordingly. This plotting method was used to examine the accuracy of the electrophoresis gels and electrophoresis results of the improved analysis platform. In this embodiment, Rf is defined as A/B, where A is the distance from the starting point of the separation gel to the protein to be identified, i.e., the distance for which the protein has migrated downward during the electrophoresis process, and B is the distance from the starting point of the separation gel to the terminal end of the separation gel, i.e., the moving distance of the dye front during the electrophoresis process. Take the example in the Bio-Rad user manual for example. The distance from the starting point to the terminal end of the separation gel is 67 mm, and the distance from the starting point of the separation gel to the protein to be identified is 45 mm; therefore, Rf is 45 mm/67 mm=0.67.

FIG. 11 shows linear fitting equations for determining the molecular weights of unknown proteins. The linear equation represented by the blue line, i.e., y=−1.544x+2.38, was established to fit all the data in the graph. The accuracy of the molecular weights determined by this equation, or more specifically the coefficient of determination of the equation, is as high as R²=0.981, although the goodness of fit is relatively low (with R²=0.969) for the small molecules (represented by the red dots). By removing the large-molecule data, the goodness of fit of the resulting fitting equation (represented by the green line, whose equation is y=−1.338x+2.254) for the small molecules is increased to R²=0.998.

The results show that both the blue-line linear equation and the green-line linear equation are highly accurate, and this demonstrates the accuracy of the gel casting and electrophoresis results of the analysis platform.

### Embodiment 7

### Blotting Performances of the Analysis Platform and of the Conventional Blotting Platform

This embodiment was a continuation of the electrophoresis results in embodiment 5.

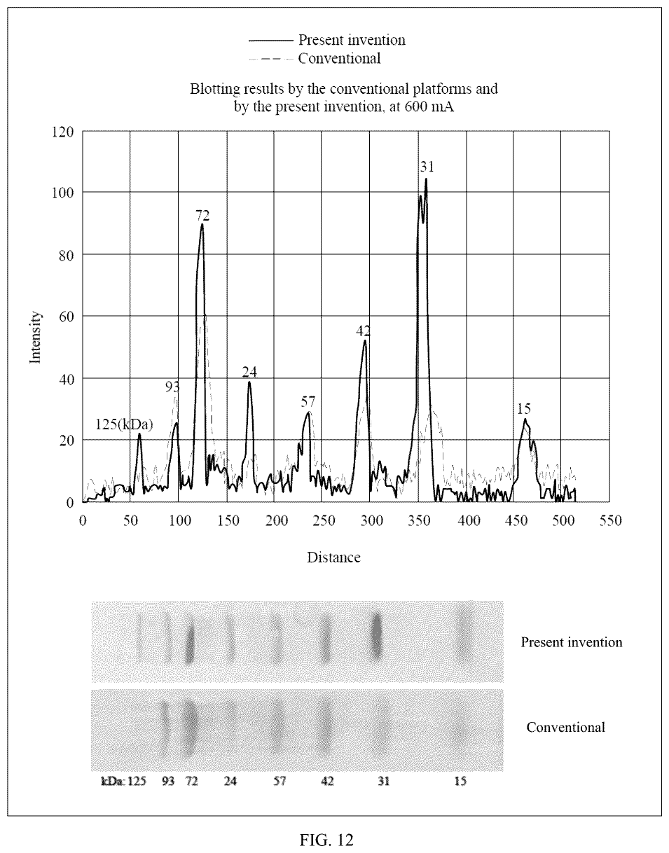

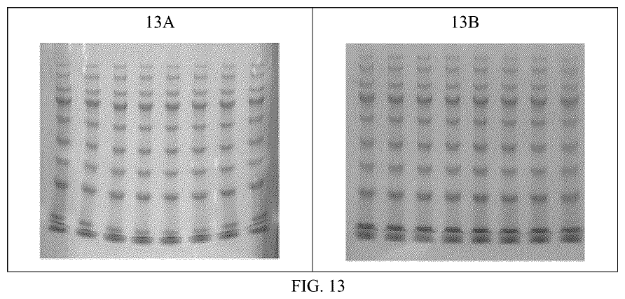

FIG. 12 shows blotting results photographed under a constant current of 600 mA at the end of a 6-minute blotting time. It can be clearly seen that the blotting result of the commercially available blotting platform has no signal band at all prior to peak 3 (which corresponds to the molecular weight of 93 kDa), and that by contrast, the platform of the present invention was able to blot molecules of 125 kDa and below on the PVDF membrane within a blotting time as short as 6 minutes. Therefore, it can be inferred that the improved analysis platform performs is better in the blotting step than the commercially available blotting platform and advantageously features a relatively short experiment time and improvement in performance. In short, the improved analysis platform can carry out wet blotting rapidly and has good blotting performance.

### Embodiment 8

### Comparison of Potential Materials of the Removable Bottom Plate

### 1. Heat Dissipation Function

Referring to FIG. 13, FIG. 13A shows the electrophoresis result obtained

by using an alumina ceramic bottom plate, and FIG. 13B shows the electrophoresis result obtained by using an aluminum nitride ceramic bottom plate. The results indicate that using the aluminum nitride ceramic bottom plate during the electrophoresis process led to straight separated bands in the gel, whereas using the alumina ceramic bottom plate resulted in separated bands with a smiling curve in the gel. It can be inferred from the above that using the aluminum nitride ceramic bottom plate during the electrophoresis process can improve the smiling curve caused by accumulation of heat, thereby allowing straight electrophoresis bands to be separated.

### 2. Contact Angle and Adhesion Force

The contact angle refers to the angle between the surface of a liquid and the solid surface on which the liquid is located, indicating the wettability of the solid surface by the liquid. The contact angle, therefore, is an important indicator in assessing the properties (e.g., roughness and chemical composition) of a solid surface and the surface tension of a liquid. When the adhesion force of a solid is greater than the cohesive force of a liquid on the solid, a relatively small contact angle can be observed, which means that the solid surface is hydrophilic: conversely, when the cohesive force is greater than the adhesion force, a relatively large contact angle can be observed, which means that the solid surface is hydrophobic.

### Method

In order to select a specific material for the removable bottom plate so that the removable bottom plate can be pulled out smoothly without dragging, and thus deforming, the gel due to excessive adhesion between the removable bottom plate and the gel, the liquid contact angles and lateral adhesion forces of removable gel-casting bottom plates made of different materials were measured. More specifically, the measurement was performed on bottom plates made of five different materials, namely an acrylic bottom plate, a glass bottom plate, an alumina ceramic bottom plate, an aluminum nitride ceramic bottom plate, and an aluminum nitride ceramic bottom plate adhesively attached with a Teflon film. The adhesion force was measured with a drop adhesion force instrument (DAFI) as follows: a platinum needle was used as the probe, the probe was spaced apart from the plate under test by a distance (H) of 300 mm and was used to drag a liquid drop (5 μL) on the plate while the deformation of the liquid drop was measured, and the deformation was converted into the magnitude of force by way of a spring constant.

### Results

The aluminum nitride ceramic bottom plate had the largest contact angle (about 119.5 to 138 degrees). Besides, the aluminum nitride ceramic bottom plate adhesively attached with a Teflon film (about 97.3 to 106.5 degrees). The glass bottom plate (not shown) had the smallest contact angle (about 15.3 to 26 degrees).

In addition, the aluminum nitride ceramic bottom plate adhesively attached with a Teflon film had a lower adhesion force than the aluminum nitride ceramic bottom plate and is therefore suitable for use as the material of the removable bottom plate.

The aforementioned results indicate that it is advantageous to use an aluminum nitride ceramic bottom plate adhesively attached with a Teflon film as the removable bottom plate.

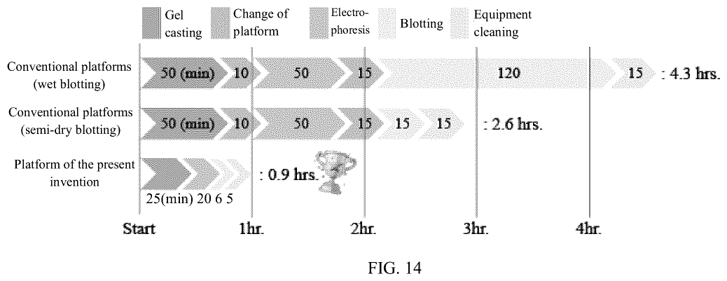

Besides, with regard to the time required for gel casting, the conventional platforms having a wet blotting function required an operation time of 4.3 hours, whereas the platform of the present invention completed the blotting process within 56 minutes, which is about 4.7 times faster than the conventional platforms having a wet blotting function and about 2.9 times faster than the conventional platforms having a semi-dry blotting function (which required 2.6 hours), as shown in FIG. 14.

According to the above, the analysis platform of the present invention integrates the functions of commercially available gel casting, electrophoresis, and blotting devices through mechanism improvement. The invention further improves the components and structures of those commercially available apparatus so as to reduce the cost required for the conventionally three discontinuous steps and for their respective equipment. Moreover, the invention allows the gel casting, electrophoresis, and blotting processes to be carried out on a single analysis platform, without having to move the gel, install additional equipment, or change equipment. More specifically, after gel casting, the user only has to bring the gel working unit into communication with the electrophoresis tanks and fill electrophoresis buffer, and the electrophoresis operation can be started without having to move the gel. Once electrophoresis is completed, there is also no need to move the gel; the user only has to remove the removable bottom plate and the upper cover plate, mount the second blotting layer stack (i.e., the upper half of the blotting sandwich), and fill blotting buffer, and blotting can be performed.

Furthermore, based on the above results, the protein analysis platform of the present invention is capable of achieving the same or even better performance in individual steps compared to commercially available devices. Thus, the invention provides an analysis platform that features high testing performance, rapidity, consistency, and high reliability. The invention also provides a use of the analysis platform in biochemical testing.

The above detailed description is a specific description of a feasible embodiment of the present invention, but the embodiment is not intended to limit the scope of the invention. Any equivalent implementation or change that does not depart from the technical spirit of the invention should be included in the scope of the invention.

1. A protein analysis platform, comprising:
a platform body comprising:
a gel working unit provided in a top portion of the platform body and comprising a gel accommodation area for accommodating at least one gel;
at least one electrophoresis tank provided along a side of the gel accommodation area and provided with at least one electrode; and
a blotting layer stack provided in a bottom portion of the gel working unit and comprising an electrode layer, wherein a removable bottom plate is provided between the gel working unit and the blotting layer stack and detachably corresponds to a bottom side of the gel accommodation area.

2. The protein analysis platform of claim 1, wherein the gel working unit further comprises an upper cover plate, and wherein the upper cover plate is detachably provided above the gel accommodation area.

3. The protein analysis platform of claim 2, wherein the gel working unit further comprises at least one upper cover plate locking element, and wherein the upper cover plate locking element is provided on at least one side of the gel working unit to press the upper cover plate toward the gel accommodation area.

4. The protein analysis platform of claim 1, wherein the gel working unit further comprises a lateral baffle plate, and wherein the lateral baffle plate is detachably provided between the gel accommodation area and the electrophoresis tank.

5. The protein analysis platform of claim 4, wherein the lateral baffle plate comprises an elastic layer provided in such a way that the elastic layer faces the gel accommodation area, and wherein the elastic layer comprises rubber, plastic, or silicone.

6. The protein analysis platform of claim 4, wherein the gel working unit further comprises at least one lateral baffle plate locking element, and wherein the lateral baffle plate locking element is provided on at least one side of the gel working unit to press the lateral baffle plate toward the gel accommodation area.

7. The protein analysis platform of claim 1, wherein the blotting layer stack further comprises a filter paper layer and a sponge layer, wherein the filter paper layer is provided above the electrode layer, and wherein the sponge layer is provided between the filter paper layer and the electrode layer.

8. The protein analysis platform of claim 1, wherein the platform body further comprises a second blotting layer stack provided above the gel working unit, wherein the second blotting layer stack comprises a second electrode layer and a polymer membrane layer, and wherein the second electrode layer is provided above the polymer membrane layer.

9. The protein analysis platform of claim 8, wherein the polymer membrane layer comprises polyvinylidene difluoride (PVDF).

10. The protein analysis platform of claim 8, wherein the second blotting layer stack further comprises a second filter paper layer and a second sponge layer, wherein the second filter paper layer is provided between the second electrode layer and the polymer membrane layer, and wherein the second sponge layer is provided between the second filter paper layer and the second electrode layer.

11. The protein analysis platform of claim 8, wherein the second blotting layer stack further comprises a blotting layer stack locking element provided above the second electrode layer and configured to be pressed toward the gel accommodation area.

12. The protein analysis platform of claim 8, wherein the electrode layer and the second electrode layer comprise platinum or titanium.

13. protein analysis platform of claim 1, wherein the removable bottom plate is a rubber plate, a plastic plate, a glass plate, a ceramic plate, or a ceramic composite substrate.

14. The protein analysis platform of claim 13, wherein the removable bottom plate is the ceramic composite substrate, wherein the ceramic composite substrate comprises a ceramic substrate and a film, wherein the film is provided on an upper surface of the ceramic substrate, and wherein the ceramic substrate comprises alumina or aluminum nitride.

15. The protein analysis platform of claim 14, wherein the film comprises polytetrafluoroethylene (PTFE).

16. The protein analysis platform of claim 1, wherein the platform body has a lateral side provided with a removable bottom plate accommodation groove for accommodating the removable bottom plate.

17. The protein analysis platform of claim 1, wherein the platform body further comprises a heat dissipation bottom plate provided under the blotting layer stack.

18. The protein analysis platform of claim 17, wherein the heat dissipation bottom plate is an alumina ceramic bottom plate, an aluminum nitride ceramic bottom plate, a thermoelectric cooling chip, or cooling fins.

19. The protein analysis platform of claim 17, wherein the platform body is peripherally provided with at least one supporting pillar for elevating the heat dissipation bottom plate.

20. A use of the protein analysis platform of claim 1, wherein the protein analysis platform is used for western blot method or a next-generation western blot method.
